(19) United States
(12) Patent Application Publication
(10) Pub. No.: US 2025/0172550 A1
(43) Pub. Date: May 29, 2025

(54) A METHOD AND SYSTEM FOR MEASURING A MODIFIED PROPERTY OF A SAMPLE COMPRISING MAGNETIC PARTICLES IN LIQUID SUSPENSION

(71) Applicants: Julius-Maximilians-Universität Würzburg, Würzburg (DE); Friedrich-Alexander-Universität Erlangen-Nürnberg, Erlangen (DE)

(72) Inventors: Patrick VOGEL, Prosselsheim (DE); Martin RÜCKERT, Reichenberg (DE); Stefan LYER, Erlangen (DE); Rainer TIETZE, Erlangen (DE); Christoph ALEXIOU, Erlangen (DE); Bernhard FRIEDRICH, Erlangen (DE)

(21) Appl. No.: 18/840,322

(22) PCT Filed: Feb. 28, 2023
(86) PCT No.: PCT/EP2023/054990

§ 371 Date: Aug. 21, 2024

(30) Foreign Application Priority Data
Feb. 28, 2022 (EP) 22159238.9

(51) Int. Cl. G01N 33/543; G01N 27/74; G01N 33/569

(52) U.S. Cl. CPC G01N 33/54333; G01N 27/745; G01N 33/54346; G01N 33/54386; G01N 33/56983; G01N 2333/165; G01N 2469/10

(57) ABSTRACT

A method for measuring a modified property of a sample comprising magnetic particles in suspension using time variable magnetic fields, wherein the method comprises applying a time-varying magnetic excitation field, the excitation field having an excitation amplitude and an excitation frequency, and an offset magnetic field to the sample, such that the magnetic particles are driven into a non-linear magnetization response regime; recording a non-linear magnetization response of the sample for obtaining a signal metric indicative for a phase of a higher harmonic with respect to the excitation frequency in the non-linear magnetization response; and determining a measurement signal indicative of the modified property, the measurement signal being based on the signal metric obtained for the sample and the same signal metric obtained for a reference sample without the modified property, the reference sample also comprising the magnetic particles in suspension.

### FIELD

The present invention is in the field of magnetic nanoparticle spectroscopy. More precisely, the present invention relates to the measurement of a modified property of magnetic micro- or nanoparticles in suspension using time-varying magnetic fields.

### BACKGROUND

The characterization of ensembles of magnetic nanoparticles (MNP) is an ongoing task in many fields of research. Magnetic particle spectroscopy (MPS) is a quite young technology for the characterization of MNPs. It uses an oscillating magnetic field of a sufficient field strength to drive a MNP ensemble periodically into a non-linear magnetization response.

The non-linear magnetization response can reveal specific information for each MNP type encoded in higher harmonics of the excitation frequency. The magnetic moments of MNPs follow the time-varying external field directions through a combined Néel and Brownian relaxation mechanism. The Brownian relaxation process is dominant for single-core iron oxide MNPs with core sizes above 20 nm and can reflect the degree of freedom of physical rotational motion of the MNPs. When the rotational degree of freedom is impeded, the non-linear magnetization response of the MNP will be affected, typically observed as a reduction of the amplitude of the higher harmonics. This property of the non-linear magnetization response can therefore enable a measurement of sample properties influencing the rotational degree of freedom of the MNPs.

For example, when the magnetic nanoparticles are suspended in liquid, the dynamic magnetic response can be used for measuring parameters of the surrounding solution, such as the viscosity and temperature, as well as the conjugations of chemical or biological compounds on the surface of the MNPs, e.g. via a modification of the MNP's hydrodynamic diameter or clustering.

Wu et al. (“One-Step, Wash-free, Nanoparticle Clustering-Based Magnetic nanoparticle Spectroscopy Bioassay Method for Detection of SARS-CoV-2 Spike and Nucleocapsid Proteins in the Liquid Phase”; ACS Appl. Mater. Interfaces 2021, 13, 44136-44146) teach a method of detecting target proteins based on polyclonal antibodies (pAbs) surface-conjugated to MNPs, such that each MNP will be able to specifically bind to target protein molecules. Samples comprising the surface-conjugated MNPs in liquid suspension as well as the target proteins are subjected to a time-varying magnetic field, and the non-linear response of the samples are compared to reference measurements of samples without the target proteins. The presence of the target proteins can be detected based on a reduction of the amplitude of higher harmonics, which can be caused by nanoparticle clustering mediated by the target proteins.

The known methods for measuring sample properties based on the dynamic magnetic response of magnetic nanoparticles are however usually reliant on accurate measurement control and require long acquisition times on the order of seconds to accurately resolve the signal amplitudes in addition to sophisticated data processing. This means that the measurement limits the sample analysis, but also that the drive currents during the measurement time can heat up the measurement device, which can lead to undesirable temperature drifts or dead time for cooling periods. Similar modalities, such as AC-susceptibility (ACS) measurement can overcome this issue offering a more sensitive technique to investigate and determine parameters of the environmental serum, but often hampers from the fact, that the handling of those devices as well as the data processing not only require sophisticated hardware but also specifically trained personnel.

In view of this state-of-the-art, the object of the invention is to provide a fast and robust method for sample characterization based on the dynamic magnetic response of magnetic micro- or nanoparticles.

### SUMMARY

This object is solved by two methods, a computer program and a system according to the independent claims. The dependent claims relate to preferred embodiments.

According to a first aspect, the invention relates to a method for measuring a modified property of a sample comprising magnetic particles in suspension using time variable magnetic fields. The method comprises applying a time-varying magnetic excitation field, the excitation field having an excitation amplitude and an excitation frequency, and a magnetic field gradient to the sample, such that the magnetic particles are driven into a non-linear magnetization response regime, and recording a non-linear magnetization response of the sample for obtaining a signal metric indicative for a phase of a higher harmonic with respect to the excitation frequency in the non-linear magnetization response. The method further comprises determining a measurement signal indicative of the modified property, the measurement signal being based on the signal metric obtained for the sample and the same signal metric obtained for a reference sample without the modified property, the reference sample also comprising the magnetic particles in suspension.

Common MPS devices are working with a strong time-varying magnetic field H_{AC}, without an additional offset field. Conversely, ACS devices are working with weak drive fields H_{AC}, whose amplitude is small enough to result in an essentially linear response of the magnetization characteristic of the magnetic particles at the excitation frequency, generally below 2 mT, and the offset magnetic fields H_{DC} (static or with low frequency with respect to the excitation frequency) excitation frequency are varied to measure the AC-susceptibility of a sample.

However, the Inventors observed that outside these two commonly measured regimes, at intermediate excitation amplitude of the time-varying magnetic excitation field and intermediate offset field, the complex signal of the higher harmonics of the excitation field experiences detectable phase shifts. These phase shifts can occur at crossing points of the real and imaginary parts of the complex signal, when plotted for a given excitation field as a function of offset magnetic field, in the following referred to as critical points. The crossing points may be located close to nodes of the signal amplitude, as predicted by the respective Chebyshev-like polynomials, and therefore at positions where, commonly, low (amplitude) signal would be expected.

However, the Inventors found that minor changes of the hydrodynamic diameter of a magnetic micro- or nanoparticle, e.g. induced by a binding event of a target analyte, can be reliably detected by tracking the phase difference between the sample and a reference sample in a wide interval around the critical point associated with the phase shift, despite the reduced signal amplitude.

The Inventors further observed that by applying a magnetic gradient field, the measurement becomes less sensitive on the exact location of the critical point for a given magnetic particle in the parameter space of offset magnetic field strength and excitation amplitude, in the following also referred to as critical point condition. Rather, it was found that the phase measurement can reliably detect minor changes of the Brownian relaxation characteristics of the magnetic particles based on the phase signal of at least one of the higher harmonics for a given gradient comprising spatially varying magnetic offset field strengths of a few mT. The Inventors found that, for small gradients, the gradient can also increase the signal-to-noise ratio of the measurement. Due to the robust nature of the measurement and since the measurement need not rely on an accurate measurement of the signal amplitude, the measurement signal can be obtained by measuring each of the sample and the reference sample once over a short measurement duration on the order of milliseconds, such that the measurement becomes suitable for fast sample characterization.

In principle, any property affecting the Brownian relaxation characteristics of the magnetic particles could be measured in this way. Additionally, the dependence of the effect on the offset magnetic field strength may also be used to measure small changes of external magnetic fields by tracking the signal metric for a sample. In other words, in some embodiments, the reference sample may be the sample measured at a different point in time, e.g. not subjected to the changed external magnetic field. However, one of the more interesting applications may be the detection of an analyte in a liquid suspension comprising the magnetic particles functionalized to bind to the analyte.

In preferred embodiments, the magnetic particles are functionalized to promote binding to a selected analyte, such that the modified property is a suspected presence of the selected analyte in the sample, wherein the analyte is in particular a biomolecule, preferably a protein, an antibody, a virus, RNA, DNA, a procariot, fungus, eucariot, or a portion thereof.

The binding of the analyte to the magnetic particles will generally change the effective hydrodynamic particle diameter, which may then influence the complex signal response of the higher harmonics, in particular by shifting the critical point condition. As a result, even small changes in hydrodynamic diameter can be detected due to the large slope associated with the phase jump at the critical point.

The magnetic particles can be prepared with functional groups to target the analytes, for example by silanizing magnetic nanoparticles using an aminosilane compound and functionalization of the resulting particle with selective binding proteins (antigen or antibodies), e.g. using suitable cross-linking agents. The resulting (functionalized) magnetic particle may selectively bind to a target biomolecule, such as a target protein of an antibody or a virus, for broad application in biochemical sensing.

In the measurement, the sample may comprise a solution associated with the suspected presence of the analyte and the reference sample may comprise a reference solution not associated with the suspected presence of the analyte, e.g. buffer solution. Both the sample and the reference sample comprise the magnetic particles functionalized to target the analyte, such that the signal metric obtained from measuring the sample and the reference sample is indicative of the suspected presence of the analyte.

The magnetic particles will generally be nano- or microparticles with a size between about 5 nm and about 10 μm, preferably with a sub-micron magnetic core, wherein the diameters of magnetic particle crystallites are such that the magnetic response is essentially superparamagnetic. For example, the magnetic particles may have a magnetic core with a diameter between about 5 nm and 1 μm and in particular between about 1 nm and about 300 nm, preferably between about 5 nm and 100 nm, and may have a shell, which may be non-magnetic and which is functionalized to bind to the target analyte.

In preferred embodiments, the magnetic field gradient and the time-varying magnetic excitation field are selected such that a range of offset magnetic field strengths of the magnetic field gradient overlaps a critical offset magnetic field range associated with a phase jump of the higher harmonic, when the sample and/or the reference sample is subjected to the excitation field.

Around the critical point condition, the phase of the higher harmonic may vary significantly, which results in an increased sensitivity to changes of the sample properties. In particular, changes of the effective hydrodynamic particle diameter may shift the critical point conditions, resulting in a large change of the phase close to the critical point.

In preferred embodiments, the phase of the higher harmonic jumps by substantially 1800 in the critical offset magnetic field range.

Generally, the phase jump associated with a critical point will have a magnitude of around 180°. However, the largest slope of the phase as a function of offset magnetic field strength may be in a range smaller than the range associated with the full phase of 180°, such as a critical offset magnetic field range in which the phase jumps by about 150°, or by about 175°. Accordingly, for an optimal sensitivity, the critical offset magnetic field range should be the range in which the majority of the phase jump is located, or a portion thereof, such that the slope of the phase as a function of offset magnetic field strength is large and a larger signal can be measured. On the other hand, a combination of excitation field and offset magnetic field gradient, which define a condition offset from the critical point may increase the dynamic range of the measurement in embodiments.

In preferred embodiments, the critical offset magnetic field range is centered on a crossing point of the real and imaginary components of the non-linear magnetization response associated with the higher harmonic.

The location of the phase jump may be determined by the crossing point of the real and imaginary components of the higher harmonic for a given excitation field, when the real and imaginary components of the higher harmonic are plotted as a function of offset magnetic field strength. The real and imaginary components of the higher harmonic may follow respective Chebyshev-like polynomials for the higher harmonic, which predict that higher harmonics of different order may have critical points located at different offset magnetic field strengths. Hence, depending on a combination of a given type of magnetic particles, a given excitation field and a given magnetic field gradient, different orders of the higher harmonics may provide a large measurement signal, for which the critical points are located in or close to the range of offset magnetic field strengths.

In some embodiments, the method may comprise selecting a specific higher harmonic for determining the measurement signal. However, since the complex amplitudes of multiple higher harmonics can be determined during digital processing of a recorded non-linear magnetization response, the selection of the higher harmonic may also be based on the measurement signal. For example, the higher harmonic associated with the highest sensitivity or the largest measurement signal may be empirically selected, or the modified property may be measured based on the measurement signals of multiple higher harmonics.

In preferred embodiments, the method comprises processing the non-linear magnetization response of the sample for obtaining complex signal amplitudes of the higher harmonics, and the signal metric is based on the complex signal amplitudes of the higher harmonic.

For example, the method may record the non-linear magnetization response as a digitized analog signal, and the digitized analog signal may be processed according to a sine and cosine transformation of a selection of higher harmonics, e.g. the first 10 or 20 higher harmonics or a select one of the higher harmonics. Based on the complex signal amplitudes of the higher harmonics, the amplitude and phase of the higher harmonics may be calculated.

In preferred embodiments, processing the non-linear magnetization response comprises a Fourier transform of the non-linear magnetization response.

The Fourier transform of the non-linear magnetization response may reveal the complex signal amplitudes of the higher harmonics and may implement the sine and cosine transformation.

Preferably, the signal metric is based on the phase determined for the higher harmonic.

In preferred embodiments, the signal metric is proportional to or mathematically equivalent to a phase of the higher harmonic.

As discussed above, the method may exploit an increased sensitivity to changes in the sample at the critical point(s), which is associated with a phase jump, such that the phase can be an effective signal metric for detecting sample changes affecting the non-linear magnetization response of the magnetic particles. However, since the crossing point is usually close to a node of the signal amplitude, at which large relative changes of the amplitude are expected, the measurement signal may also be determined based on a function of the complex signal amplitudes, e.g. by including a measurement signal contribution of the amplitude and the phase. In other words, the measurement signal may merely be indicative for a phase difference of the higher harmonic for the sample and for the reference sample.

The condition for the offset magnetic field strength at which a critical point is located may depend on the components of the magnetic field gradient oriented along the direction of the excitation field.

In preferred embodiments, the excitation field induces a magnetic field having an excitation field direction, and wherein a parallel component of the magnetic field gradient along the excitation field direction varies over a measurement volume associated with the sample and/or the reference sample.

By varying the offset magnetic field strength over the measurement volume, the critical point condition may be fulfilled in at least a portion of the sample or the reference sample.

In preferred embodiments, the magnetic field gradient comprises offset magnetic field strengths larger than 2 mT.

The inventors found in their experiments with iron oxide magnetic particles that the phase jumps are observed for offset magnetic field strengths above 2 mT, preferably larger than 2.5 mT.

Although a detectable measurement signal should also be present at lower offset magnetic fields according to the simulations of the Inventors, offset magnetic field strengths above 2 mT may facilitate implementing the method without sophisticated measurement hardware. As an example, the offset magnetic field strengths of the magnetic field gradient in the measurement volume may be centered on a value between about 2 mT and about 40 mT, such as between 2 mT and 20 mT or between 2 mT and 10 mT.

The range of offset magnetic field strengths of the magnetic field gradient in the measurement volume may be selected based on a desired compromise between a robustness of the method with respect to the critical point conditions and a magnitude of the measurement signal, which may benefit from a smaller width of the magnetic field gradient. As an example, the range of offset magnetic field strengths of the magnetic field gradient in the measurement volume may be on the order of between about 0.1 mT to about 5 mT, such as about 0.5 mT, about 1 mT, or about 2 mT.

In other words, a variation of the offset magnetic field in the measurement volume based on the magnetic field gradient may be larger than 0.1 mT, such as larger than 0.5 mT, larger than 1 mT, or larger than 2 mT around a mean value between about 2 mT and about 40 mT. For example, a component of the offset magnetic field may vary in a range of offset magnetic fields of between about 0.1 mT to about 5 mT over the (reference) sample around a mean value between about 2 mT and about 40 mT.

In preferred embodiments, applying the magnetic field gradient comprises applying the field of a permanent magnet.

The permanent magnet may generate a substantially static magnetic field gradient in the measurement volume and may contribute to the robustness of the method. The permanent magnet may be offset from the measurement volume, in particular offset in a direction perpendicular to the direction of the excitation field, for inducing a suitable gradient across the measurement volume. The permanent magnet may induce offset magnetic fields, which need not align with excitation direction associated with the time-varying magnetic excitation field. The skilled person will appreciate that in these cases, the critical point condition is substantially selected based on the magnetic field components of the magnetic field gradient parallel to the excitation direction. The offset fields may further be tuned using an additional homogenous magnetic field, e.g. applied with a magnetic field coil, in order to select the critical point condition for the sample and/or the reference sample.

In preferred embodiments, the excitation field has an excitation amplitude larger than 2 mT.

Similar to the value of the offset magnetic field strength for the critical point condition, the Inventors observed a strong measurement signal in their experiments for critical point conditions at excitation amplitudes, which are larger than 2 mT, preferably larger than 2.5 mT. In particular, the excitation amplitudes may be outside of the linear range of the magnetization characteristic of the magnetic particles. The simulations of the Inventors also predict a measurement signal for lower excitation amplitudes. However, based on the empirical measurement results, an excitation field amplitude above about 2 mT may facilitate implementing the method without sophisticated measurement hardware.

In preferred embodiments, the measurement signal is mathematically equivalent or proportional to a difference between the signal metric obtained for the sample and the signal metric obtained for the reference sample.

For example, the phase of the higher harmonics may be recorded for the reference sample and the sample, and the resulting signal metrics may be subtracted from each other as a phase difference between a higher harmonic of the sample and of the reference sample.

In preferred embodiments, the method further comprises applying the excitation field and the magnetic field gradient to the reference sample without the modified property, and recording a non-linear magnetization response of the reference sample for obtaining the signal metric of the higher harmonic for the reference sample.

The sample and the reference sample may be measured in parallel or in succession for obtaining respective signal metrics for determining the measurement signal indicative of the modified property. However, the reference sample may also be measured and the resulting signal metric may be recorded and compared to respective signal metrics of a plurality of different samples in sample characterization applications measured at different points in time.

The measurement signal may be determined on a processing system. The processing system may comprise a single processing unit or may comprise a plurality of processing units, which may be functionally connected. The processing units may comprise a microcontroller, an ASIC, a PLA (CPLA), an FPGA, or other processing device, including processing devices operating based on software, hardware, firmware, or a combination thereof. The processing devices can include an integrated memory, or communicate with an external memory, or both, and may further comprise interfaces for connecting to sensors, devices, appliances, integrated logic circuits, other controllers, or the like, wherein the interfaces may be configured to receive or send signals, such as electrical signals, optical signals, wireless signals, acoustic signals, or the like. For example, the processing system may be connected to an alternating current generator for driving an excitation coil to apply the time-varying magnetic field and/or may be coupled to an analog to digital converter for recording a current induced in a measurement coil to record the non-linear magnetization response of the sample. The processing system may also be configured to determine the signal metric of the higher harmonic based on the recorded non-linear magnetization response, e.g. by implementing a fast Fourier transform of the recorded non-linear magnetization response.

According to a second aspect, the invention relates to a further method for measuring a modified property of a sample comprising magnetic particles in suspension using time variable magnetic fields. The method comprises applying a time-varying magnetic excitation field, the excitation field having an excitation amplitude and an excitation frequency, and a variable offset magnetic field to the sample, such that the magnetic particles are driven into a non-linear magnetization response regime, and recording a non-linear magnetization response of the sample for obtaining a signal metric indicative for a phase of a higher harmonic with respect to the excitation frequency in the non-linear magnetization response. The method further comprises determining a measurement signal indicative of the modified property, the measurement signal being based on the signal metric obtained for the sample and the signal metric obtained for a reference sample without the modified property, the reference sample also comprising the magnetic particles in suspension.

In preferred embodiments, the variable offset magnetic field is selected from a critical offset magnetic field range associated with a phase jump of the higher harmonic, when the sample and/or the reference sample is subjected to the excitation field.

Hence, the method may also be implemented by selecting the critical point condition using an excitation field having a suitable excitation amplitude and a suitable non-zero offset magnetic field. The resulting method may be more susceptible to instrumentation noise and may require more sophisticated measurement hardware, but may also attain a higher measurement signal, as the critical point condition may be fulfilled in a larger portion of the sample volume, when compared to the use of a comparatively large magnetic field gradient.

The offset magnetic field may be generated with a magnetic field coil. The offset magnetic field may be substantially static when compared to the excitation frequency and/or when compared to an acquisition time of the non-linear magnetization response.

In preferred embodiments, the offset magnetic field and/or the excitation amplitude are varied across the phase jump of the higher harmonic for the sample and/or the reference sample, while recording the non-linear magnetization response of the sample to the time-varying magnetic excitation field, wherein the measurement signal is preferably based on a resulting trace of the signal metric for the sample and a corresponding trace of the signal metric for the reference sample.

In other words, the critical point condition may be scanned by varying the excitation amplitude and/or the offset magnetic field in order to select the critical point condition. For example, the offset magnetic field may be varied in steps while the sample is subjected to the excitation field, for recording the nonlinear magnetization response close to a critical point condition of the higher harmonic. After the characterization, it may be sufficient to measure a single value of the signal metric for the sample and the reference sample.

The method according to the second aspect may be combined with the method according to the first aspect, e.g. may comprise applying a magnetic field gradient in addition to a variable offset magnetic field, such as a variable offset magnetic field generated with a magnetic field coil for tuning the measurement system. Further, the method according to the second aspect may also benefit from any feature of the preferred embodiments of the first aspect.

According to a third aspect, the invention relates to a measurement system for measuring a modified property of a sample comprising magnetic particles in suspension using time variable magnetic fields. The system comprises an inductive sensor associated with a measurement volume, a magnetic field coil for generating a time-varying magnetic field in the measurement volume, and an offset magnetic field source for generating an offset magnetic field in the measurement volume, and a control system. The control system is configured to drive the magnetic field coil to apply a time-varying magnetic excitation field to the measurement volume, the excitation field having an excitation amplitude and an excitation frequency, such that the magnetic particles of the sample are driven into a non-linear magnetization response regime, and receive a non-linear magnetization response of the sample from the inductive sensor for obtaining a signal metric indicative for a phase of a higher harmonic with respect to the excitation frequency in the non-linear magnetization response. The control system is further configured to determine a measurement signal indicative of the modified property, the measurement signal being based on the signal metric obtained for the sample and the signal metric obtained for a reference sample without the modified property, the reference sample also comprising the magnetic particles in suspension.

The system may implement the method according to the first aspect or the second aspect or any combination of their embodiments.

In preferred embodiments, the offset magnetic field and the time-varying magnetic excitation field are selected, such that the offset magnetic field is in a critical offset magnetic field range associated with a phase jump of the higher harmonic, when the sample and/or the reference sample is subjected to the excitation field.

In preferred embodiments, the magnetic field source comprises a variable magnetic field generator for generating the offset magnetic field in the critical offset magnetic field range.

The variable magnetic field generator may be a constant current source coupled to a coil for inducing an offset magnetic field in the measurement volume. The variable magnetic field generator may generate a substantially homogeneous magnetic field in the measurement volume for selecting the critical point condition in a large portion of the sample volume.

In preferred embodiments, the variable magnetic field generator is coupled to the magnetic field coil or to a second magnetic field coil.

In preferred embodiments, the variable magnetic field generator is configured to vary the offset magnetic field over the critical offset magnetic field range for the sample and the reference sample, while the system records the non-linear magnetization response to the time-varying magnetic excitation field.

In preferred embodiments, the magnetic field source is adapted to induce a magnetic field gradient in the measurement volume and in particular comprises a permanent magnet arranged to induce a magnetic field gradient in the measurement volume.

The magnetic field gradient induced by the permanent magnet may comprise the offset magnetic field in the critical offset magnetic field range.

However, the skilled person will appreciate that a magnetic field gradient may also be implemented dynamically using suitable magnetic field coils, or magnetic field coils and permanent magnets may be combined to implement a range of offset magnetic fields overlapping the critical offset magnetic field range.

In preferred embodiments, the inductive sensor comprises a measurement coil, in particular a gradiometer.

The gradiometer may comprise two magnetometers in series for measuring a magnetic flux difference between the magnetometers, and the sample may be arranged in one of the magnetometers during a measurement. The gradiometer may reduce a contribution of the excitation field in the recorded magnetization response and may therefore simplify an acquisition hardware. However, the system may equally reduce a corresponding signal contribution using measurement hardware, e.g. using suitable high-pass filtering.

According to a fourth aspect, the invention relates to a non-transitory medium comprising machine-readable instruction, which, when executed by a processing system, implement a method according to the first aspect and/or the second aspect, or a system according to the third aspect.

### BRIEF DESCRIPTION OF THE DRAWINGS

The features and numerous advantages of the methods and system according to the present invention will best be understood from a detailed description of preferred embodiments with reference to the accompanying drawings, in which:

FIG. 1 schematically illustrates the non-linear magnetization response of a sample comprising magnetic nanoparticles in liquid suspension;

FIG. 2A, 2B illustrate an example of the real and imaginary parts as well as the phase of the third harmonic as a function of offset magnetic field strength;

FIG. 3A shows an example of the measured real and imaginary parts as well as the phase of the third harmonic as a function of offset magnetic field strength;

FIG. 3B illustrates the phase of the third harmonic as a function of both excitation field amplitude and offset magnetic field strength in a grey-scale color plot;

FIG. 4 illustrates an example of a method for measuring a modified property of a sample comprising magnetic nanoparticles in suspension using time variable magnetic fields;

FIG. 5 illustrates a system for implementing the method of FIG. 4;

FIG. 6 illustrates an example of a nanoparticle functionalization sequence for targeting a specific analyte, based on a SARS CoV-2-S1 protein;

FIGS. 7A-D illustrate example measurements of the phase difference of the third harmonic between two pairs of samples;

FIGS. 8A, 8B illustrate examples of measurements of the phase difference between two respective pairs of samples as a function of offset magnetic field and excitation field amplitude for a plurality of different higher harmonics;

FIG. 9A schematically illustrates an example of a system similar to the system of FIG. 5, further comprising a permanent magnet for inducing a magnetic field gradient in the measurement volume;

FIG. 9B schematically illustrates an example of a distribution of the offset magnetic field components projected onto the excitation field direction in a system as shown in FIG. 9A;

FIG. 10 illustrates an example of a method for measuring a modified property of a sample comprising magnetic nanoparticles in suspension using a magnetic field gradient;

FIG. 11A, 11B illustrate raw data of measurements using a system as illustrated in FIG. 9A based on a method as illustrated in FIG. 10; and

FIGS. 12A-C illustrate measurements of the presence of an analyte at different concentrations based on the phase difference of the 7^{th} and 9^{th} higher harmonics as well as based on fluorescence activated cell sorting, respectively.

### DETAILED DESCRIPTION

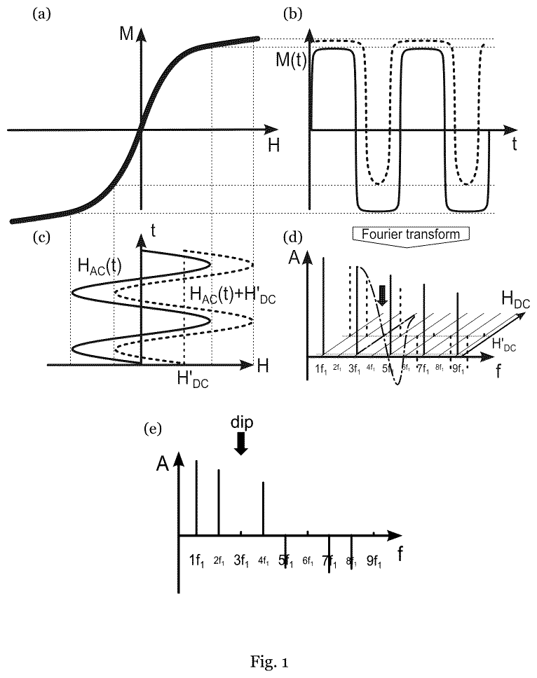

FIG. 1 schematically illustrates the non-linear magnetization response of a sample comprising magnetic nanoparticles in liquid suspension, such as iron oxide nanoparticles with a crystallite diameter between about 5 nm and about 100 nm in a liquid suspension.

In absence of an external magnetic field, all magnetic nanoparticles in such a sample may be statistically oriented, which may cause the magnetization of the sample to be zero. Increasing the external magnetic field strength can lead to more and more particles aligning along the external field and cause an increase of the magnetization. At a specific magnetic field strength Mₛₐₜ, all particles may be aligned and the magnetization of the sample is saturated (saturation magnetization).

The dependency of the sample magnetization M to the external magnetic field strength H may be effectively superparamagnetic and can be described by the Langevin function L(ξ):

$$L ( ξ ) = coth ( ξ ) - \frac{1}{ξ} with ξ = \frac{{μ}_{0} mH}{{k}_{B} T} , \qquad ( 1 )$$

with m as the magnetic moment of a particle, μ₀ as the vacuum permeability, kB as the Boltzmann constant and T as temperature. The resulting curve is shown in subfigure FIG. 1(a), showing a substantially linear response regime around zero external magnetic field H as well as a saturation regime, in which the slope of the magnetization with respect to the external magnetic field strength H is reduced.

When the sample is subjected to a time-varying field H_{AC}(t) with an excitation frequency f_{AC} and a substantially static offset magnetic field H_{DC} with sufficient amplitude, the magnetic nanoparticles can be driven into a non-linear response regime, in which the magnetization M(t) is no longer a linear function of the external magnetic field strength H.

Subfigures FIG. 1(c) and FIG. 1(b) show two different driving fields with identical sinusoidal time-varying field components H_{AC}(t) components and the resulting time dependent magnetization response, respectively, for the case of zero offset magnetic field (solid curves), and non-zero offset magnetic field H_{DC}′ (dashed curves). Dashed lines between subfigures FIG. 1(a)-(c) illustrate the magnitude of the magnetic field strength and of the resulting magnetization response in relation to the magnetization curve in FIG. 1(a).

As can be seen from FIG. 1(b), the magnetization response M(t) of the sample to a time-varying field H_{AC}(t) (H_{AC}(t)+H_{DC}′) with a sufficient magnitude differs from a sinusoidal shape.

Rather, the magnetization response the magnetic nanoparticles becomes non-linear and exhibits higher harmonics of the excitation frequency f_{AC} in frequency domain.

FIG. 1(d) shows an example of a Fourier spectrum of the signals of FIG. 1(b). The horizontal axis is frequency, the vertical axis shows the amplitude of the real part of the Fourier transform A of the magnetization response, and the perpendicular axis indicates offset magnetic field H_{DC}. FIG. 1(e) shows the dependency of the real part of the Fourier transform A as a function of frequency for the example with offset magnetic field H_{DC}′ (corresponding to the dashed curves of FIGS. 1(b)-(d)).

As can be seen in the Fourier spectrum associated with offset magnetic field H_{DC}′ illustrated in FIG. 1(e), the magnetization response M(t) consists not only of the fundamental frequency but also odd (and even) higher harmonics (f&=n-fi) depending on the presence of an offset magnetic field H_{DC}, which can be visualized in the Fourier spectrum.

The Fourier spectrum further shows a complex distribution of the signal amplitude among the different higher harmonics, where in the exemplified example, the third harmonic, which can be measured at frequency 3·f_{AC} (“3fi”), shows a signal amplitude close to zero (“dip”). The amplitude Aₙ of the higher harmonics for a given time-varying excitation field with excitation amplitude H_{AC} as a function of offset magnetic field H_{DC} strength may be predicted using the Chebyshev polynomials according to

$${A}_{n} ( {H}_{DC} ) = - \frac{2 i}{T} {M}^{′} ( {H}_{DC} ) * ( {U}_{n - 1} ( \frac{{H}_{DC}}{{H}_{AC}} ) \cdot \sqrt{1 - {( \frac{{H}_{DC}}{{H}_{AC}} )}^{2}} ) \qquad ( 2 )$$

where Uₙ₋₁ represents the Chebyshev polynomials of the second kind and M′(H_{DC}) is the derivative of the magnetic response, which is exemplified for the third order higher harmonic by the curve in FIG. 1(d) with intermittent dashes (-·-).

In the signal, the ‘dips’ within a Fourier spectrum of a MPS signal may be understood as a destructive interference of two separate signal components Sₚₒₛ and S_{neg} associated with respective switches of the magnetization in opposite directions, which arise from the time signal S(t)=dM(t)/dt, which is captured as an induction signal by a receive coil. At the condition of the dips H_{DC}, the signals Sₚₒₛ and S_{neg} associated with a higher harmonic may have similar amplitude, but may effectively cancel each other due to a phase shift between the signals Sₚₒₛ and S_{neg}. Said phase shift may be offset magnetic field dependent due to a shift of the points in time at which the sample is not in a saturation regime (as can be seen from subfigure FIG. 1 (b)). As a result of the interference, the phase of the higher harmonic signal may experience a phase jump close to the conditions for the “dip”.

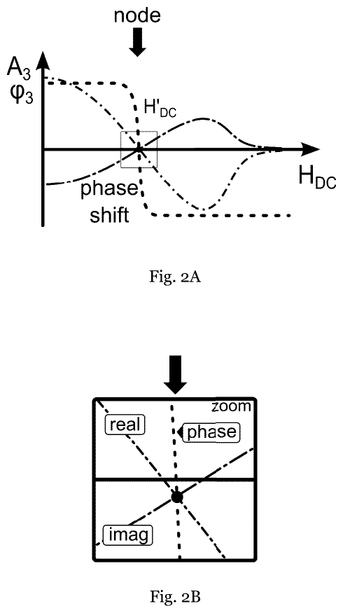

FIG. 2A illustrates an example of the real and imaginary parts of the amplitude A₃ as well as the phase φ3 of the third harmonic as a function of offset magnetic field strength H_{DC}. FIG. 2B illustrates a zoom of the square portion of FIG. 2A around the offset magnetic field strength H_{DC}′.

As can be seen from the diagram, the phase φ3 of the third harmonic shifts by 180° at the crossing of the real part and the imaginary part of the amplitude. The crossing is close to, but shifted from the nodes (zeros) of the real part and the imaginary part of the amplitude A₃. The slope of the phase φ3 with respect to offset magnetic field strength H_{DC} is larger than the slope of the real part and the imaginary part of the amplitude A₃ between respective extremal values.

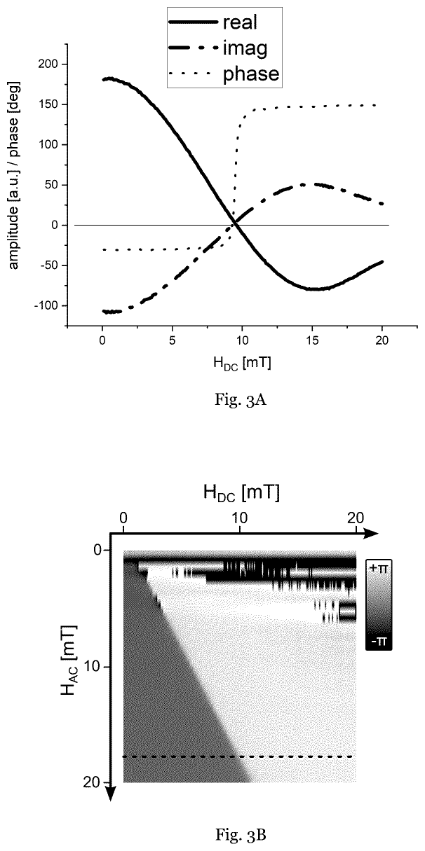

FIG. 3A shows an example of the measured real and imaginary parts of the amplitude A₃ as well as the phase φ3 of the third harmonic in an experiment with functionalized magnetic iron oxide nanoparticles in liquid suspension, as a function of offset magnetic field strength H_{DC}. FIG. 3B illustrates the phase φ3 of the third harmonic as a function of both excitation field amplitude H_{AC} and offset magnetic field strength H_{DC} in a grey-scale color plot, wherein the curve in FIG. 3B is highlighted as a dashed line through the visualization.

As can be seen from the measured data, the phase φ3 shifts abruptly by substantially 180° over an offset magnetic field range of about 2,5 mT, in accordance with the theoretical prediction at the crossing of the real part and the imaginary part of the amplitude (critical point condition). The phase jump is pronounced in the data shown in FIG. 3B, starting at excitation field amplitudes H_{AC} of about 2 mT and offset magnetic field H_{DC} of at least 1.5 mT, and may be clearly identified for critical point conditions with excitation field amplitudes H_{AC} above about 2 mT and offset magnetic fields H_{DC} above about 2.5 mT. The excitation field amplitude H_{AC} is preferably greater than the offset magnetic field H_{DC}.

The Inventors propose to exploit the strong slope associated with the phase shift around the critical point condition for these intermediate values of excitation field amplitudes H_{AC} and offset magnetic field H_{DC} as a sensing platform for detecting minor changes in sample properties affecting the effective hydrodynamic diameter of the magnetic nanoparticles, in particular to sense binding events to the magnetic nanoparticles.

In a liquid suspension, the magnetization response m(t) not only depends on the magnetic properties of the magnetic nanoparticle's particles, but also on the effective viscosity experienced by the magnetic nanoparticles in the liquid medium. Specifically, the magnetization response m to an external magnetic field H in a liquid medium may be described by

$$\frac{d \vec{m}}{dt} = \frac{1}{ζ} ( \vec{m} \times \vec{H} ) \times \vec{m} + \sqrt{\frac{2 {k}_{B} T}{ζ}} \vec{λ} \times \vec{m} , \qquad ( 3 )$$

with ζ as the Stokes-Einstein diffusion coefficient (viscosity-coefficient) and \vec{λ} as normally distributed random vectors with expectation value <λᵢ(t)>=0, k_{B} as the Boltzmann constant and T as the temperature. The diffusion coefficient may be approximated according to ζ=κηR³ and may depend on the viscosity q of the surrounding liquid, the particle diameter R and a shape factor κ.

Hence, a binding event to the magnetic nanoparticle, which may affect the effective particle diameter, may shift the critical point condition of the higher harmonics, which may be tracked based on the complex signal of the higher harmonics, in particular a signal metric based on the phase (e.g. the phase φ₃) of the higher harmonic.

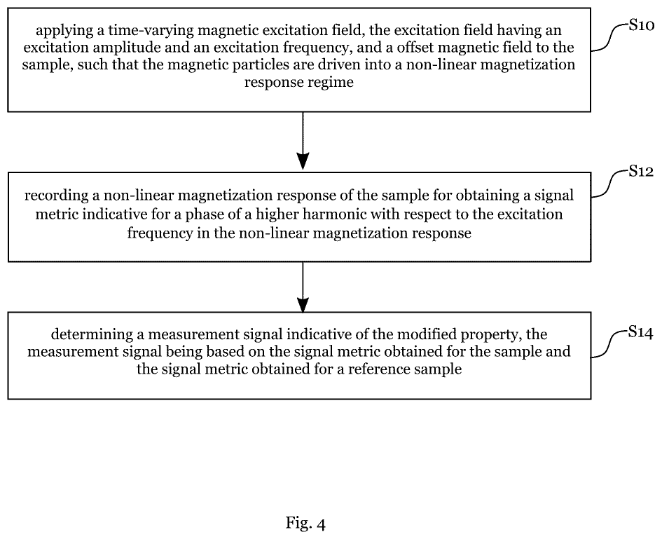

FIG. 4 illustrates an example of a method for measuring a modified property of a sample comprising magnetic particles in suspension using time variable magnetic fields. The method comprises applying a time-varying magnetic excitation field H_{AC}(t), the excitation field having an excitation amplitude H_{AC} and an excitation frequency f_{AC}, and a offset magnetic field H_{DC} to the sample, such that the magnetic particles are driven into a non-linear magnetization response regime (S10), and recording a non-linear magnetization response of the sample for obtaining a signal metric indicative for a phase of a higher harmonic with respect to the excitation frequency f_{AC} in the non-linear magnetization response (S12). The method further comprises determining a measurement signal indicative of the modified property, the measurement signal being based on the signal metric obtained for the sample and the signal metric obtained for a reference sample (S14).

The offset magnetic field H_{DC} is selected from a critical offset magnetic field range associated with a phase jump of the higher harmonic, when the sample and/or the reference sample is subjected to the excitation field, which may generally correspond to the afore-mentioned critical point condition. The critical offset magnetic field range may be a range of offset magnetic fields H_{DC} around the critical point condition, in which the phase jumps by substantially 180°, such as by 120° more, or 160° or more, and the method may therefore be sensitive to small changes of the particle parameters. However, the skilled person will appreciate that any sub-range of the critical offset magnetic field range may equally be used to implement the method in some embodiments. The reference sample also comprises the magnetic particles in suspension, but does not feature the modified property.

The (reference) sample may be characterized as in FIG. 3A/3B for identifying the critical point condition, such that further samples may be measured with substantially fixed values of the offset magnetic field H_{DC} and the excitation amplitude H_{AC}. Additionally or alternatively, one or both of the offset magnetic field H_{DC} and the excitation amplitude H_{AC} may be varied to obtain curves of the phase as a function of offset magnetic field H_{DC} and/or excitation amplitude H_{AC} for each sample. A comparison of the measured signal metrics for the reference sample and for the sample may then indicate the (suspected) modified property.

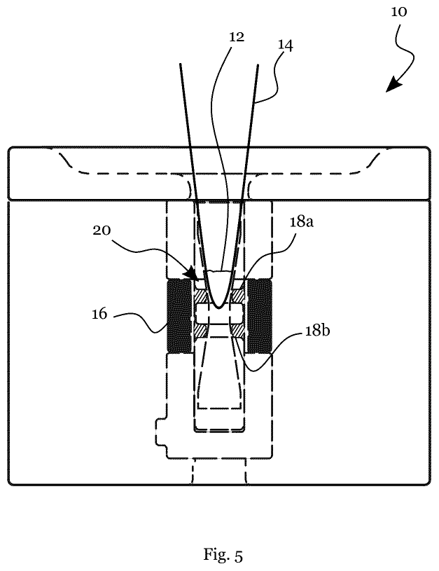

FIG. 5 illustrates a system 10 for measuring a (reference) sample 12 in a liquid container 14. The system 10 comprises an excitation field coil 16 and a detection coil 18a, 18b implemented as a gradiometer comprising winding sections 18a, 18b of opposite winding direction. The signals induced in the two winding sections 18a, 18b having opposite winding directions from the excitation coil 16 may cancel each other, when the resulting added signal is measured. Conversely, signals from (small) magnetization changes of the sample 12 in a measurement volume 20 associated with one of the winding sections 18a are not cancelled. In other words, the current of the winding sections 18a, 18b may be proportional to a magnetic flux difference between the one of the winding sections 18a and the other winding section 18b, while common magnetic flux components may be cancelled. Accordingly, small induction signals from the sample 12 may be measured without sophisticated measurement hardware.

The excitation field coil 16 may be driven with a combination of static and time-varying currents for generating the offset magnetic field H_{DC} and the excitation field H_{AC}(t), but some embodiments of the system 10 may also comprise different coils 16 for generating the offset magnetic field H_{DC} and the excitation field H_{AC}(t), respectively.

Multiple sample containers 14 may be prepared with different (reference) samples 12 and may be swapped for different measurements, during which the current sample 12 comprising the magnetic particles is subjected to the offset magnetic field H_{DC} and the excitation field H_{AC}(t), while recording the non-linear magnetization response from the measurement volume 20. The signal from the detection coil 18a, 18b may be amplified and digitized, and a Fourier transform of the digitized non-linear magnetization response may be used to obtain the complex amplitudes of the higher harmonics.

To measure a presence of a specific analyte in the sample 12, magnetic nanoparticles may be functionalized, such that they bind selectively to the specific analyte. The binding of the analyte to the magnetic nanoparticles may be associated with a corresponding change in the hydrodynamic diameter of the magnetic nanoparticles (MNPs), and may therefore be associated with a measurable change of the critical point condition.

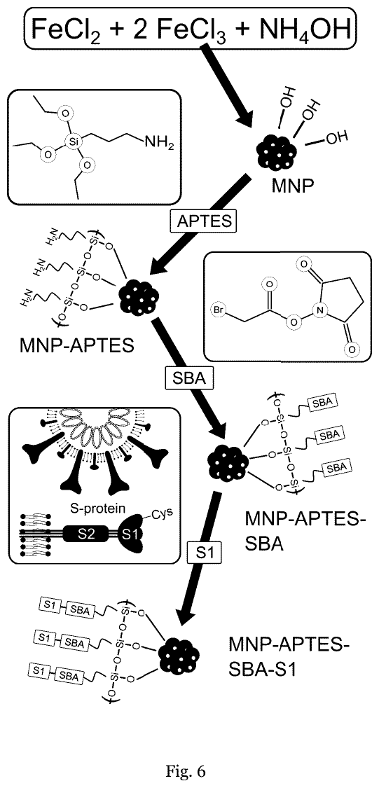

FIG. 6 illustrates an example of a nanoparticle functionalization strategy for targeting a specific analyte, based on a SARS CoV-2-S1 protein. After alkaline precipitation of the raw particles, the exemplary sequence comprises an initial silanization step, in which MNPs may be coated with APTES ((3-aminopropyl) triethoxysilane). The MNPs produced in this way may have a hydrodynamic diameter of about 200 nm with the single crystallites of the magnetic nanoparticle core showing a diameter of about 12 nm.

SARS-CoV-2-S1 protein (SARS-CoV-2 (2019-nCoV) spike S1-His, Sino Biological, China) can be covalently bound to the surface of the particles by binding SBA (N-succinimidyl bromoacetate) over cysteines present in the protein. In the experiments of the Inventors, a 0.05 M borate buffer with pH 8.5 was used for binding. The particle concentration during processing was adjusted to 1 mg Fe/ml and 20 mM SBA dissolved in Dimethylformamide was added. The samples 12 were shaken for 2 h at 1400 rpm. After that, the particles were washed several times with buffer solution. The obtained particles were redispersed in borate buffer for binding of SARS-CoV-2-S1 protein with a concentration of 10 g S1 per 100 g Fe. The samples 12 were shaken again for 2 h at 1400 rpm and then washed several times with doubly distilled H2O. After binding and the last washing step, the hydrodynamic size of the particles was 330 nm and the particles were stored in doubly distilled water.

The SARS-CoV-2-S1 protein may selectively bind to SARS-CoV-2-S1 antibody as an analyte, and the magnetic nanoparticles in a sample 12 may therefore be used to detect the presence of the analyte through a change of the hydrodynamic diameter associated with the analyte bound to the magnetic nanoparticles.

For the following example measurements, for each (reference) sample 12, 25 μL of MNP-APTES-S1 dispersions (100 g Fe/mL) was added in an 0.5 mL Eppendorf cap. This magnetic nanoparticle solution was combined with a buffer solution (reference sample), an S1 binding antibody (SARS-CoV-2-S1 antibody) solution (binding sample, S+) or a non-binding antibody (MERS-CoV-S1 antibody) solution (non-binding sample, S−), and measured without any further incubation time. The (reference) samples 12 are excited with an excitation current featuring a time-varying modulation at an excitation frequency of about 20 kHz applied to the excitation coil 16 and the non-linear magnetization response from the sample 12 may be recorded in a detection coil 18a, 18b, amplified, and digitized for extracting the complex signal amplitudes associated with the higher harmonics.

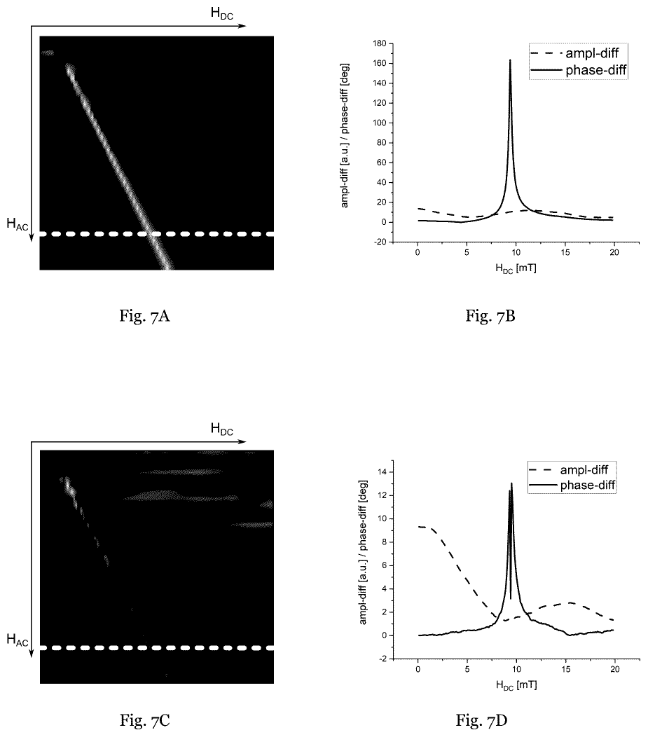

FIGS. 7A-D illustrate example measurements of the phase difference |δφ| of the third harmonic between two pairs of samples 12 as a function of offset magnetic field H_{DC}. FIGS. 7A, 7C show the dependency on excitation field amplitude H_{AC} in a greyscale color plot, whereas FIGS. 7B, 7D, show line traces at fixed excitation field amplitude H_{AC} along the white dashed lines. The axes of the greyscale color plot correspond to the axes of the measurement shown in FIG. 3B, i.e. the offset magnetic field H_{DC} is varied along the horizontal axis from 0 to 20 mT (from left to right), and the excitation amplitude H_{AC} is varied along the vertical axis from 0 to 20 mT (from top to bottom). The magnitude of the phase difference |δφ| in FIGS. 7B, 7D is in the same arbitrary units, and may therefore be compared quantitatively. FIGS. 7A, 7B show the phase difference |δφ| PI between a binding sample 12 and a reference sample 12, while FIGS. 7A, 7B show the phase difference measured between two reference samples 12.

As shown in FIG. 7B, the phase difference |δφ| measured between the binding sample 12 and the reference sample 12 shows a pronounced peak at an offset magnetic field strength H_{DC} of about 9 mT, which may be attributed to a shift of the critical point condition caused by the presence of the binding antibody. This peak may be attributed to a modified effective hydrodynamic diameter of the magnetic nanoparticles in the sample 12 and the phase difference at the magnetic field conditions of the peak may therefore be used to detect a suspected presence of the analyte.

As can be seen from FIGS. 7A, the presence of the analyte can be detected based on the phase difference |δφ| for a variety of combinations of excitation field amplitude H_{AC} and offset magnetic field strength H_{DC}. Specifically, for the third harmonic the phase difference |δφ| is associated with a visible peak around critical point conditions with an excitation field amplitude H_{AC} above around 2.5 mT and an offset magnetic field strength H_{DC} above around 2 mT, corresponding substantially to the parameters associated with a detectable phase jump in FIG. 3B.

As shown in FIG. 7D, the same measurements also result in a peak of the phase difference |δφ| at substantially the same magnetic field conditions, when two reference samples 12 are compared, which the Inventors attribute to systematic errors and instrumentation noise. However, the magnitude of the phase difference |δφ| visible in FIGS. 7B, 7D differs by an order of magnitude from the phase difference |δφ| measured between the binding sample 12 and the reference sample 12, indicating that a corresponding measurement can be used to reliably detect the presence of the binding antibody.

Each measurement of the non-linear magnetization response may in principle be analyzed for obtaining the complex amplitudes and phases of a plurality of higher harmonics, and the signals associated with each of the higher harmonics may in principle be exploited for detecting the presence of the analyte (e.g. target antibody).

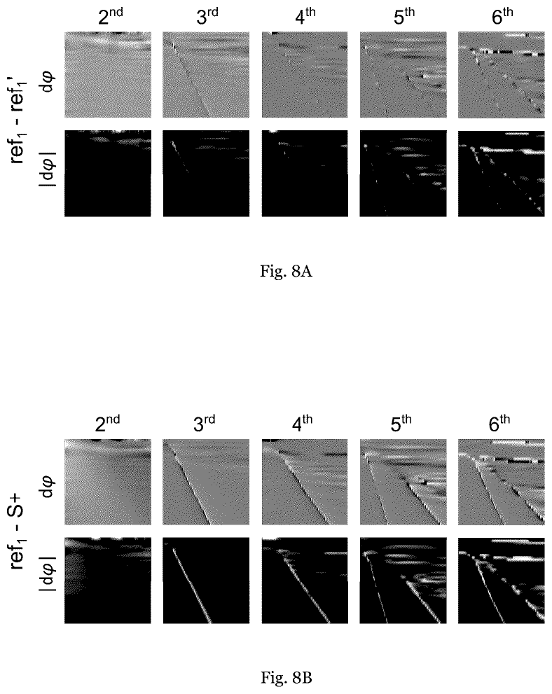

FIGS. 8A, 8B illustrate examples of measurements of the phase difference |δφ| between two samples 12 as a function of offset magnetic field strength H_{DC} and excitation field amplitude H_{AC} for a plurality of different higher harmonics. FIG. 8A shows the measured phase difference |δφ| between two reference samples 12 (ref, ref′), while FIG. 8B shows the measured phase difference |δφ| between a reference sample 12 (ref) and a binding sample 12 (S+). The phase signal of different higher harmonics are identified by their order (“2^{nd}, 3^{rd}, 4^{th}, 5^{th}, 6^{th}”) for the second to sixth higher harmonic. The axes of each plot are the same as the ones of FIG. 7A, 7C.

The comparison of the data illustrates that the presence of the analyte may be reliably detected based on the measured phase difference |δφ| for all of the higher harmonics starting from the 3^{rd} higher harmonic, while the location of the critical point condition associated with the phase jump in the data differs for different higher harmonics.

Hence, by selecting a suitable combination of offset magnetic field strength H_{DC} and excitation field amplitude H_{AC} a precise measurement method may be implemented on the basis of measurement signals from different higher harmonics.

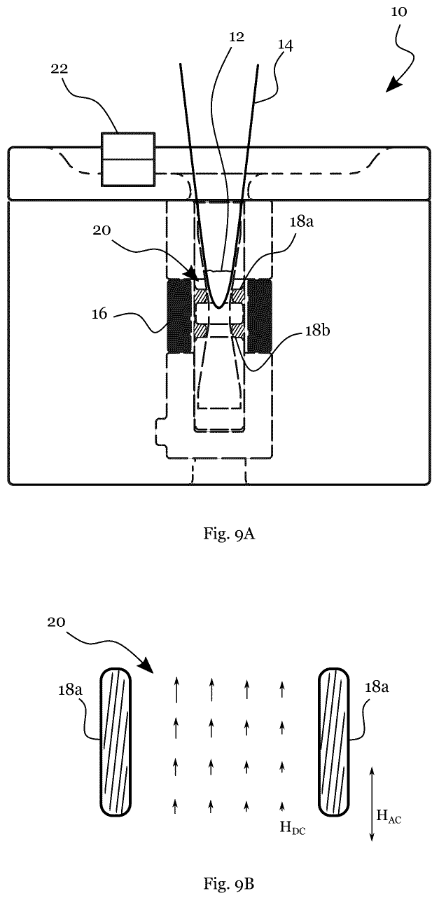

FIG. 9A illustrates a further system for a system 10 for measuring a (reference) sample 12 in a liquid container 14, which differs from the system illustrated in FIG. 6 by the addition of a permanent magnet 22. FIG. 9B illustrates a schematic example of the spatial distribution of the offset magnetic field strength H_{DC} component induced by the permanent magnet 22 (magnitude of offset magnetic field indicated by differently sized arrows), which is oriented along the excitation field direction (indicated schematically by double-headed arrow in FIG. 9B), i.e. resulting from a projection of the respective magnetic field strengths onto the excitation field direction.

The permanent magnet 22 is arranged with an offset from the detection coil 18a, 18b and the excitation coil 16, in a direction along an excitation field direction (indicated schematically by double-headed arrow in FIG. 9B) of the excitation field generated by the excitation coil 16, and in a direction perpendicular to the excitation field direction. As a result, the offset magnetic field strength H_{DC} in the measurement volume 20 associated with the one of the winding sections 18a may vary spatially as illustrated in the example in FIG. 9B.

The spatially varying offset magnetic field strength H_{DC} induced by the permanent magnet 22 in the measurement volume 20 may therefore define an offset magnetic field gradient, which may lead to the critical point condition being fulfilled in a portion of a (reference) sample 12 20 for a given excitation field H_{AC}. The offset magnetic field gradient may reduce a requirement of sophistication related to the measurement of a modified property of the sample 12, as the critical point condition may be fulfilled in a broader range of critical offset magnetic fields. In some embodiments, the permanent magnet 22 may be the only offset magnetic field source, such that no direct current may be required for selecting the critical point condition.

For example, the system 10 may comprise a neodymium permanent magnet 22, which may induce magnetic field strengths in a range between about 5 mT to about 10 mT in the measurement volume 20. Hence, a component of the offset magnetic field H_{DC} along the excitation field direction (i.e. the illustrated z-projection) may vary in a range of offset magnetic fields of about 3 mT over the (reference) sample 12 around a mean value of about 5 mT.

However, the permanent magnet 22 may also be combined with additional (in-)homogenous offset magnetic fields H_{DC}, e.g. generated with a magnetic field coil 16, in some embodiments.

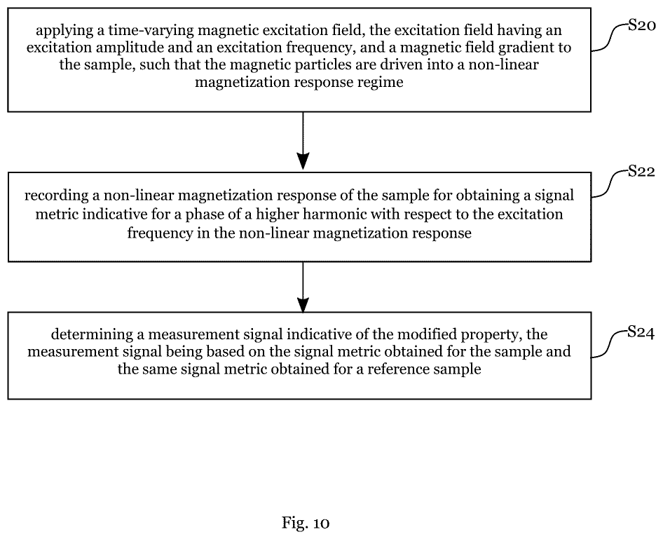

FIG. 10 illustrates an example of a method for measuring a modified property of a sample 12 using a magnetic field gradient. The method comprises applying a time-varying magnetic excitation field H_{AC}(t), the excitation field having an excitation amplitude H_{AC} and an excitation frequency f_{AC}, and a magnetic field gradient to the sample 12, such that the magnetic particles are driven into a non-linear magnetization response regime (S20), and recording a non-linear magnetization response of the sample 12 for obtaining a signal metric indicative for a phase of a higher harmonic with respect to the excitation frequency f_{AC} in the non-linear magnetization response (S22). The method further comprises determining a measurement signal indicative of the modified property, the measurement signal being based on the signal metric obtained for the sample 12 and the same signal metric obtained for a reference sample 12 (S24).

The reference sample 12 does not feature the modified property, but also comprises the magnetic particles in suspension. The magnetic field gradient and the time-varying magnetic excitation field H_{AC}(t) are selected such that a range of offset magnetic field strengths H_{DC} of the magnetic field gradient overlaps a critical offset magnetic field range associated with a phase jump of the higher harmonic, when the sample 12 and/or the reference sample 12 is subjected to the excitation field H_{AC}(t).

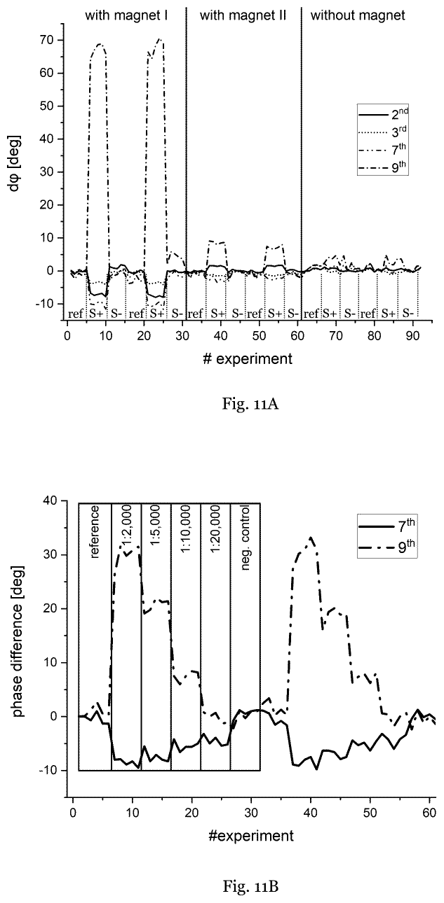

FIG. 11A, 11B illustrate raw data of preliminary measurements using a system 10 with a permanent magnet 22 inducing a magnetic field gradient in a measurement volume 20 as illustrated in FIG. 9A based on a method as illustrated in FIG. 10. The horizontal axis indicates an identifier associated with an experiment of a sequence of consecutive experiments conducted in chronological order, while the vertical axis illustrates a measured phase difference |δφ| between a measurement of a sample 12 and a reference sample 12 (e.g. measured at the start of the sequence), wherein the phase difference |δφ| is exemplarily shown for three different higher harmonics, as specified in the legend.

Each phase measurement is the result of a Fourier analysis of the non-linear magnetization response of the respective (reference) samples 12 to an applied time-varying magnetic excitation field H_{AC}(t) at an excitation frequency f_{AC} of 20 kHz and an excitation amplitude H_{AC} of approximately 50 mT over a duration of approx. 10 ms, while it is affected by the offset magnetic field gradient of the permanent magnet 22 in the measurement volume 20.

The samples 12 were changed after a number of consecutive measurements of the phase difference |δφ|, wherein the respective samples 12 are indicated in the lower portion of FIG. 11A. In chronological order, the samples 12 were cycled between a reference sample 12 (“ref”) containing a buffer solution, a binding sample 12 (“S+”) containing a binding antibody in the buffer solution, and a non-binding sample 12 (“S−”) containing a non-binding antibody in the buffer solution, as specified above.

After 30 measurements with the permanent magnet 22 in a first configuration (“with magnet I”), the orientation of the permanent magnet 22 was rotated by 90° towards a second configuration (“with magnet II”), which effectively results in a reduction of the component of the offset magnetic field strength H_{DC} parallel to the excitation field direction in the measurement volume 20. After 60 measurements, the permanent magnet 22 was removed from the vicinity of the measurement volume 20 (“without magnet”), such that the offset magnetic field strength H_{DC} substantially vanishes in the measurement volume 20.

For the first configuration (“with magnet I”), the phase of all but the third illustrated higher harmonics shows a clear signal which is distinguished both from a corresponding measurement of a reference sample 12 and a non-binding sample 12, while the phase difference is largest for the ninth (“9^{th}”) higher harmonic. For the second configuration, the measurement shows significantly reduced magnitude of the phase difference |δφ| for all of the illustrated higher harmonics, but a presence can still be detected based on the phase difference |δφ| measured for the ninth (“9^{th}”) higher harmonic. The Inventors attribute the sensitivity of the measurement based on the phase difference |δφ| of the ninth (“9^{th}”) higher harmonic in the second configuration on the low offset magnetic field strength H_{DC} for which the critical point condition is fulfilled for this higher harmonic. Accordingly, although the overlap with the respective critical offset magnetic field range is reduced, the measurement remains sensitive to the presence of the binding antibodies. In both configurations of the permanent magnet 22, the binding sample 12 (“S+”) may be distinguished from the non-binding sample 12 (“S−”) based on the measured phase difference |δφ|.

When the magnet is removed (“without magnet”), the method can no longer distinguish between a binding sample 12 (“S+”) and the reference sample 12 (“ref”) or the non-binding sample 12 (“S−”) based on the measured phase difference |δφ|. This behavior is expected, as this configuration does not select a critical point condition for measuring the presence of the binding antibody in the sample 12 based on the resulting offset magnetic field H_{DC} of substantially zero.

The skilled person will appreciate that the phase difference |δφ| of the second (“2^{nd}”) higher harmonic equally shows a clear signal for the binding sample 12 (“S+”) with respect to the non-binding sample 12 (“S−”) and the reference sample 12 (“ref”), although no phase jump was visible in the data of FIG. 8A, 8B. The Inventors attribute the sensitivity of the phase difference |δφ| of the second (“2^{nd}”) higher harmonic to a phase jump around zero offset magnetic field H_{DC}, predicted for all even higher harmonics, wherein the magnetic field gradient selects portions of the critical offset magnetic field range associated with this phase jump close to zero offset magnetic field H_{DC}. Hence, although the amplitude of the even higher harmonics substantially vanishes for zero offset magnetic field, the phase of the corresponding signal at comparatively low offset magnetic fields may still be sensitive to the presence of the binding antibody. However, as also visible in FIG. 11A, 11B and confirmed by the measurements displayed in FIGS. 8A, 8B, a measurement signal associated with critical points (phase jumps) at non-zero offset magnetic field can achieve a sensitivity which is approximately an order of magnitude larger than the sensitivity obtained based on the critical point around zero offset magnetic field, which is measured for the second (even) higher harmonic. The phase difference |δφ| of the second (“2^{nd}”) higher harmonic also substantially vanishes in the absence of the permanent magnet 22 (“without magnet”).

FIG. 11B shows similar experiments as in FIG. 11A, but in addition to a reference sample 12 (“ref”), and a non-binding sample (“neg. control”), further binding samples (“1:2,000, 1:5,000, 1:10,000, 1:20,000”) having different dilutions of the binding antibody solution are measured. The sample 12 with a dilution of 1:20,000 of antibody solution to buffer solution corresponds to an antibody concentration of 50 ng/ml, resulting in a sensitivity of about 2 BAU (binding antibody unit).

The data shows an approximate proportionality between antibody concentration and the measured phase difference |δφ| for the binding sample 12, which may indicate a suitability of the method for quantitative characterization of antibody concentrations in the sample 12. Moreover, the method appears to discriminate between the reference/non-binding sample 12 and the binding sample 12 also at dilutions of the antibody solution of about 1:20,000 for the 7^{th} higher harmonic.

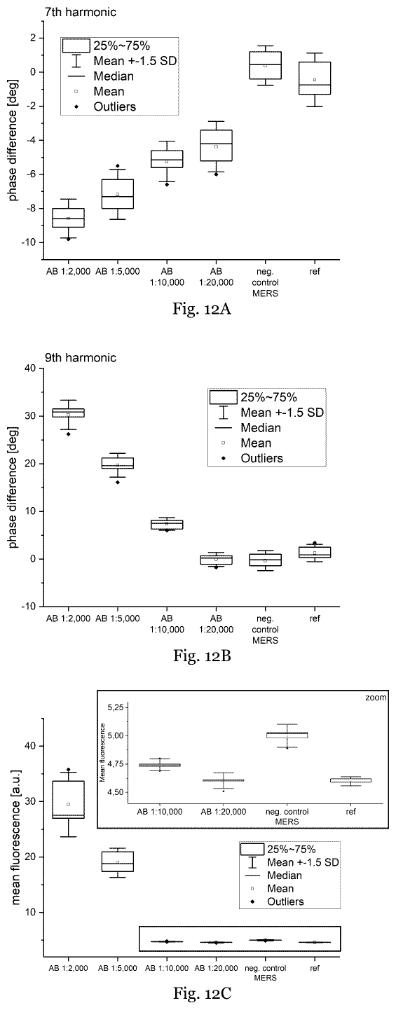

FIGS. 12A, 12B illustrate the resulting median and mean values of the phase difference |δφ| (horizontal axis) associated with the measurement sequences of a given reference/binding/non-binding sample 12 including statistical error bars for the measurement results of five consecutive measurements of each sample 12 (as specified in the horizontal axis). The statistical analysis confirms a sensitivity of the measurement to the binding antibodies up to dilutions of 1:10,000 for the 9^{th} higher harmonic (FIG. 12A) and up to dilutions of 1:20,000 for the 7^{th} higher harmonic (FIG. 12B).

The reduced sensitivity of the 9^{th} higher harmonic may be a result of a decaying complex signal magnitude for higher orders of the higher harmonics and/or related to the limited sampling frequency of the system 10 used for the preliminary experiments, and may therefore likely be overcome with more sophisticated measurement hardware.

FIG. 12C illustrates a comparative measurement of identically prepared samples 12 as in FIGS. 12A, 12B using fluorescence activated cell sorting (also known as FACS or “Flow cytometry”). The inset shows the mean fluorescence measured for each sample 12. Detection of antibodies was possible up to a dilution of 1:5000 with FACS-analysis.

As a result, the afore-mentioned methods may be exploited to implement a robust and fast sample 12 characterization method with a performance at least comparable to state of the art biochemical sensing methods, such as ELISA (Enzyme-linked Immunosorbent Assay) with a sensitivity of about 1-2 BAU. The results are particularly promising since the excitation amplitude H_{AC} was not optimized to select a specific phase jump, but the selected condition only partially overlaps a critical offset magnetic field range of the 7^{th} and 9^{th} higher harmonics. Further improvements related to the sensitivity are therefore expected for lower excitation amplitudes H_{AC} or larger offset magnetic fields H_{DC}, e.g. using stronger permanent magnets 22. On the other hand, the measurements in FIGS. 11A-12B illustrate that the precise selection of the critical condition is not necessary for implementing a corresponding measurement. Rather, the presence of a non-zero offset magnetic field (gradient) H_{DC} in combination with a suitable excitation amplitude to drive the magnetic nanoparticles into a non-linear magnetization response can suffice to measure the presence of the binding antibody, e.g. by selecting a “tail” of the critical offset magnetic field range.

The magnetic particles may be made of any suitable compound showing superparamagnetic behavior in a liquid suspension. Commonly, the magnetic core of magnetic nanoparticles comprises magnetite (Fe3O4) or maghemite (γ-Fe2O3) crystallites, which may feature a substantially fixed magnetization direction for crystallite diameters smaller than 100 nm. However, larger particle diameters may equally be used, e.g. as a result of larger nanoparticles being usually composed of multiple crystallites each showing essentially superparamagnetic properties. Such larger diameters may also be favored in some applications for facilitating a surface functionalization. Further, the diameter of the magnetic nanoparticles may also be increased due to the surface functionalization. For example, a functionalized magnetic nanoparticle may have a diameter of around 300 nm as discussed above. Accordingly, the skilled person will appreciate that the magnetic nanoparticles may have diameters of more than 100 nm or more than 300 nm in some embodiments, such as between 5 nm and 1000 nm or between 5 nm and 10 μm, and the magnetic particles may cluster because of binding, which may further increase the particle size.

Although the preferred embodiments have been described in conjunction with a preferred application related to biochemical sensing, other applications may equally be contemplated, wherein a modified property of the sample affects the rotational degree of freedom of the magnetic nanoparticles in suspension. For example, the method may be used to track a temperature of a sample 12 based on associated viscosity changes of a liquid suspension. As a further example, since the phase can be highly sensitive to offset magnetic field, the Inventors also tested the method for measuring changes of magnetic field, and found that the resolution for magnetic field changes can be on the order of μT without significant optimization. Hence, the reference sample 12 may also be the sample 12 measured at a different point in time.

For sensing applications of magnetic fields, the magnetic (nano-)particles may also be smaller, such that the magnetic response may be significantly affected by Néel relaxation, since the magnetic particles do not necessarily have to be functionalized. Moreover, the skilled person will appreciate that for Néel relaxation the magnetic particles may also not perform a physical rotation. However, the phase jump will still be present for the critical point condition and may be used to measure changes of magnetic fields.

In this case, the excitation frequency may also be higher, e.g. more than 100 kHz, when compared to the common excitation frequencies for measuring particles in which the Brown relaxation effects results in a comparatively well-resolved inductive signal, such as between 1 kHz and 100 kHz.

The description of the preferred embodiments and the figures merely serve to illustrate the invention and the beneficial effects associated therewith, but should not be understood to imply any limitation. The scope of the invention is to be determined solely by the appended claims.

### LIST OF REFERENCE SIGNS

system
12 sample
14 sample container
16 excitation coil
18a, 18b upper and lower detection coil windings of gradiometer configuration
20 measurement volume
22 permanent magnet

What is claimed is:

1-25. (canceled)

26. A method for measuring a modified property of a sample comprising magnetic particles in suspension using time variable magnetic fields, wherein the method comprises:
applying a time-varying magnetic excitation field, the excitation field having an excitation amplitude and an excitation frequency, and a magnetic field gradient to the sample, such that the magnetic particles are driven into a non-linear magnetization response regime;
recording a non-linear magnetization response of the sample for obtaining a signal metric indicative for a phase of a higher harmonic with respect to the excitation frequency in the non-linear magnetization response; and
determining a measurement signal indicative of the modified property, the measurement signal being based on the signal metric obtained for the sample and the same signal metric obtained for a reference sample without the modified property, the reference sample also comprising the magnetic particles in suspension.

27. The method of claim 26, wherein the magnetic particles are functionalized to promote binding to a selected analyte, such that the modified property is a suspected presence of the selected analyte in the sample wherein the analyte is a biomolecule, a protein, an antibody, a virus, RNA, DNA, a procariot, fungus, eucariot, or a portion thereof.

28. The method of claim 26, wherein the magnetic field gradient and the time-varying magnetic excitation field are selected such that a range of offset magnetic field strengths of the magnetic field gradient overlaps a critical offset magnetic field range associated with a phase jump of the higher harmonic, when the sample or the reference sample is subjected to the excitation field.

29. The method of claim 28, wherein the phase of the higher harmonic jumps by substantially 1800 in the critical offset magnetic field range.

30. The method of claim 28, wherein the critical offset magnetic field range is centered on a crossing point of the real and imaginary components of the non-linear magnetization response associated with the higher harmonic.

31. The method of claim 26, wherein the method comprises processing the non-linear magnetization response of the sample for obtaining complex signal amplitudes of the higher harmonics, and wherein the signal metric is based on the complex signal amplitudes of the higher harmonic.

32. The method of claim 26, wherein processing the non-linear magnetization response comprises a Fourier transform of the non-linear magnetization response.

33. The method of claim 26, wherein the signal metric is proportional to or mathematically equivalent to a phase of the higher harmonic.

34. The method of claim 26, wherein the excitation field induces a magnetic field having an excitation field direction, and wherein a parallel component of the magnetic field gradient along the excitation field direction varies over a measurement volume associated with the sample or the reference sample, wherein applying the magnetic field gradient comprises applying the field of a permanent magnet, and
wherein the magnetic field gradient comprises offset magnetic field strengths larger than 2 mT.

35. The method of claim 26, wherein the excitation field has an excitation amplitude larger than 2 mT.

36. The method of claim 26, wherein the measurement signal is mathematically equivalent or proportional to a difference between the signal metric obtained for the sample and the signal metric obtained for the reference sample.

37. The method of claim 26, wherein the method further comprises:
applying the excitation field and the magnetic field gradient to the reference sample without the modified property;
recording a non-linear magnetization response of the reference sample for obtaining the signal metric indicative for the phase of the higher harmonic for the reference sample.

38. A method for measuring a modified property of a sample comprising magnetic particles in suspension using time variable magnetic fields, wherein the method comprises:
applying a time-varying magnetic excitation field, the excitation field having an excitation amplitude and an excitation frequency, and a variable offset magnetic field to the sample, such that the magnetic particles are driven into a non-linear magnetization response regime;
recording a non-linear magnetization response of the sample for obtaining a signal metric indicative for a phase of a higher harmonic with respect to the excitation frequency in the non-linear magnetization response; and
determining a measurement signal indicative of the modified property, the measurement signal being based on the signal metric obtained for the sample and the signal metric obtained for a reference sample without the modified property, the reference sample also comprising the magnetic particles in suspension.

39. The method of claim 38, wherein the offset magnetic field is selected from a critical offset magnetic field range associated with a phase jump of the higher harmonic, when the sample or the reference sample is subjected to the excitation field.

40. The method of claim 39, wherein the offset magnetic field or the excitation amplitude are varied across the phase jump of the higher harmonic for the sample or the reference sample, while recording the non-linear magnetization response of the sample to the time-varying magnetic excitation field, wherein the measurement signal is based on a resulting trace of the signal metric for the sample and a corresponding trace of the signal metric for the reference sample.

41. A measurement system for measuring a modified property of a sample comprising magnetic particles in suspension using time variable magnetic fields, wherein the system comprises:
an inductive sensor associated with a measurement volume;
a magnetic field coil for generating a time-varying magnetic field in the measurement volume;
an offset magnetic field source for generating an offset magnetic field in the measurement volume; and
a control system, wherein the control system is configured to:
drive the magnetic field coil to apply a time-varying magnetic excitation field to the measurement volume, the excitation field having an excitation amplitude and an excitation frequency, such that the magnetic particles of the sample are driven into a non-linear magnetization response regime, and
receive a non-linear magnetization response of the sample from the inductive sensor for obtaining a signal metric indicative for a phase of a higher harmonic with respect to the excitation frequency in the non-linear magnetization response;
determine a measurement signal indicative of the modified property, the measurement signal being based on the signal metric obtained for the sample and the signal metric obtained for a reference sample without the modified property, the reference sample also comprising the magnetic particles in suspension.

42. The system of claim 41, wherein the offset magnetic field and the time-varying magnetic excitation field are selected, such that the offset magnetic field is in a critical offset magnetic field range associated with a phase jump of the higher harmonic, when the sample or the reference sample is subjected to the excitation field.

43. The system of claim 42, the magnetic field source comprising a variable magnetic field generator for generating the offset magnetic field in the critical offset magnetic field range, and
wherein the variable magnetic field generator is coupled to the magnetic field coil or to a second magnetic field coil.

44. The system of claim 43, wherein the variable magnetic field generator is configured to vary the offset magnetic field over the critical offset magnetic field range for the sample and the reference sample, while the system records the non-linear magnetization response to the time-varying magnetic excitation field.

45. The system of any one of claim 41, wherein the magnetic field source is adapted to induce a magnetic field gradient in the measurement volume and comprises a permanent magnet arranged to induce a magnetic field gradient in the measurement volume.
